(19) United States
(12) Patent Application Publication
(10) Pub. No.: US 2023/0340079 A1
(43) Pub. Date: Oct. 26, 2023

(54) METHODS OF IMPROVING PROTEIN EXPRESSION

(71) Applicant: ASTRAZENECA AB, Södertälje (SE)

(72) Inventors: Emma KELSALL, Cambridge (GB); Suzanne GIBSON, Cambridge (GB); Diane HATTON, Cambridge (GB)

(21) Appl. No.: 18/001,643

(22) PCT Filed: Jul. 2, 2021
(86) PCT No.: PCT/IB2021/055960

§ 371 Date: Dec. 13, 2022

Related U.S. Application Data
(60) Provisional application No. 63/049,380, filed on Jul. 8, 2020.
(60) Provisional application No. 63/211,648, filed on Jun. 17, 2021.

(51) Int. Cl. C07K 16/06

(52) U.S. Cl. CPC C07K 16/06; C07K 2317/10

(57) ABSTRACT

The present disclosure relates to nucleic acids that comprise a nucleotide sequence encoding an immunoglobulin heavy chain, wherein the nucleotide sequences of at one or two introns in the immunoglobulin heavy chain are deleted. These nucleic acids are useful for increasing immunoglobulin expression.

### FIELD OF THE DISCLOSURE

The present disclosure relates to improved methods of expressing polypeptides of interest. Nucleic acids that comprise a nucleotide sequence encoding an immunoglobulin heavy chain and intron deletions are useful to increase specific cellular productivity of immunoglobulins.

### BACKGROUND

DNA is made up of intronic and exonic sequences, with introns removed during mRNA processing by splicing. This process is closely linked to mRNA export out of the nucleus through the nuclear pore complexes. This export is fundamental for expression and is well documented in the literature. See Kohler A. et al., Nature Review Molecular Cell Biology 8:761-773 (2007); Bjork, P. et al., Seminars in Cell & Developmental Biology 32:47-54 (2014); Reed, R. Current Opinion in Cell Biology, 15:326-331 (2003).

It has been observed that the presence of introns within the codon-optimized heavy chain constant region encoded in expression vectors result in an improvement in harvest titer compared to the same nucleotide sequence without introns in this region. However, the risk associated with using expression vectors containing introns is that intron-retention and mis-splicing events can occur during expression of proteins of interest, such as antibodies or immunoglobulins, resulting in unwanted aberrant protein species that need to be removed during purification. This adds extra complexity to the purification process and potential batch-to-batch variation if these alternative species cannot be removed during downstream processes.

It has been shown that using a non-codon optimized sequence containing the introns from the heavy chain constant region can eliminate the occurrence of splice variants in the heavy chain constant region. The nucleotide sequence changes occurring from codon optimization is thought to have introduced cryptic splicing resulting in these alternative species. Also, it has been shown that removing each one of the introns individually (thereby leaving two of the three introns remaining) shows a surprising improvement in harvest titer.

Removal of one or two introns from the immunoglobulin heavy chain constant region can reduce the risk of intron-retention and mis-splicing events, while increasing immunoglobulin expression. This will result in a new generation of expression vectors, which pose a low risk of generating aberrant protein species during production of antibody- or immunoglobulin-based recombinant proteins.

### SUMMARY

The present disclosure is generally directed an isolated nucleic acid comprising a nucleotide sequence encoding an immunoglobulin heavy chain, wherein the nucleotide sequences of intron 2 and intron 3 of the immunoglobulin heavy chain constant region are deleted.

The present disclosure is also directed to an isolated nucleic acid comprising a nucleotide sequence encoding an immunoglobulin heavy chain wherein the nucleotide sequence of one intron of the immunoglobulin heavy chain constant region is deleted. In one aspect, the nucleotide sequence of intron 1 of the immunoglobulin heavy chain constant region is deleted. In another aspect, the nucleotide sequence of intron 2 of the immunoglobulin heavy chain constant region is deleted. In another aspect, the nucleotide sequence of intron 3 of the immunoglobulin heavy chain constant region is deleted.

The present disclosure is also directed to an isolated nucleic acid comprising a nucleotide sequence encoding an immunoglobulin heavy chain, wherein the nucleotide sequences of two introns of the immunoglobulin heavy chain constant region are deleted. In one aspect, the nucleotide sequences of intron 1 and intron 2 of the immunoglobulin heavy chain constant region are deleted. In another aspect, the nucleotide sequences of intron 1 and intron 3 of the immunoglobulin heavy chain constant region are deleted.

The present disclosure is also directed to an isolated nucleic acid comprising a nucleotide sequence encoding an immunoglobulin heavy chain, wherein the nucleotide sequence of intron 1 of the immunoglobulin heavy chain constant region is deleted and the nucleotide sequence of intron 2 and/or intron 3 of the immunoglobulin heavy chain constant region are deleted, and wherein the nucleotide sequences of intron 2 and/or intron 3 are substituted with the nucleotide sequence of intron 1. In one aspect, the nucleotide sequence of intron 2 of the immunoglobulin heavy chain constant region is substituted with the nucleotide sequence of intron 1. In one aspect, the nucleotide sequence of intron 3 of the immunoglobulin heavy chain constant region is substituted with the nucleotide sequence of intron 1.

The present disclosure is also directed to an isolated nucleic acid comprising a nucleotide sequence encoding an immunoglobulin heavy chain, wherein the nucleotide sequences of intron 2 and/or intron 3 of the immunoglobulin heavy chain constant region are replaced with the nucleotide sequence of intron 1. In one aspect, the the nucleotide sequence of intron 2 of the immunoglobulin heavy chain constant region is replaced with the nucleotide sequence of intron 1. In one aspect, the nucleotide sequence of intron 3 of the immunoglobulin heavy chain constant region is replaced with the nucleotide sequence of intron 1.

The present disclosure is also directed to an isolated nucleic acid comprising a nucleotide sequence encoding an immunoglobulin heavy chain, wherein the nucleotide sequence of intron 3 of the immunoglobulin heavy chain constant region is replaced with a nucleotide sequence of an intron comprising about the same number of nucleotides as the nucleotide sequence of intron 1 of the immunoglobulin heavy chain constant region.

The present disclosure is also directed to an isolated nucleic acid comprising a nucleotide sequence encoding an immunoglobulin heavy chain, wherein the nucleotide sequence of intron 2 of the immunoglobulin heavy chain constant region is replaced with a nucleotide sequence of an intron comprising about the same number of nucleotides as the nucleotide sequence of intron 1 of the immunoglobulin heavy chain constant region. In one aspect, the nucleotide sequence of intron 1 of the immunoglobulin heavy chain constant region is deleted.

In one aspect, the nucleic acid expresses an immunoglobulin at a higher titer than a nucleic acid containing all intron sequences of the immunoglobulin heavy chain constant region when expressed with a nucleic acid encoding an immunoglobulin light chain. In another aspect, the nucleic acid expresses an immunoglobulin at a higher titer than a nucleic acid containing no intron sequences of the immunoglobulin heavy chain constant region when expressed with a nucleic acid encoding an immunoglobulin light chain. In another aspect, the immunoglobulin light chain is a kappa light chain or lambda light chain. In another aspect, the nucleic acid is codon optimized. In another aspect, the expressed immunoglobulin has an IgG1, IgG2, IgG3, or IgG4 isotype. In another aspect, the expressed immunoglobulin is a human, humanized, chimeric, or resurfaced immunoglobulin. In another aspect, the nucleic acid encoding a immunoglobulin heavy chain is a deoxyribonucleic acid (DNA).

In another aspect, the disclosure is directed to a vector or expression vector comprising a nucleic acid of the disclosure. In another aspect, the disclosure is directed to a host cell comprising a vector or expression vector of the disclosure. In one aspect, the host cell is a eukaryotic cell, for example a Chinese hamster ovary (CHO) cell.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequences of intron 2 and intron 3 of the immunoglobulin heavy chain constant region are deleted and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequence of one intron of the immunoglobulin heavy chain constant region is deleted and a nucleic acid encoding an immunoglobulin light chain, wherein the host cell expresses the immunoglobulin at a higher titer than a host cell comprising a nucleic acid encoding an immunoglobulin heavy chain comprising all or none of introns 1-3 of the immunoglobulin heavy chain constant region are present, and a nucleic acid encoding an immunoglobulin light chain. In one aspect, the nucleotide sequence of intron 1 of the immunoglobulin heavy chain constant region is deleted. In another aspect, the nucleotide sequence of intron 2 of the immunoglobulin heavy chain constant region is deleted. In another aspect, the nucleotide sequence of intron 3 of the immunoglobulin heavy chain constant region is deleted.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequences of two introns of the immunoglobulin heavy chain constant region are deleted and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain. In one aspect, introns 1 and 2 of the immunoglobulin heavy chain constant region are deleted. In another aspect, introns 1 and 3 of the immunoglobulin heavy chain constant region are deleted.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequence of intron 1 of the immunoglobulin heavy chain constant region is deleted and the nucleotide sequence of intron 2 and/or intron 3 of the immunoglobulin heavy chain are deleted, and wherein the nucleotide sequences of intron 2 and/or intron 3 are substituted with the nucleotide sequence of intron 1 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 the immunoglobulin heavy chain constant region are present, and a nucleic acid encoding an immunoglobulin light chain. In one aspect, the nucleotide sequence of intron 2 of the immunoglobulin heavy chain constant region is substituted with the nucleotide sequence of intron 1. In another aspect, the nucleotide sequence of intron 3 of the immunoglobulin heavy chain constant region is substituted with the nucleotide sequence of intron 1.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequences of intron 2 and/or intron 3 of the immunoglobulin heavy chain constant region are replaced with the nucleotide sequence of intron 1 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 the immunoglobulin heavy chain constant region are present, and a nucleic acid encoding an immunoglobulin light chain. In one aspect, the nucleotide sequence of intron 2 of the immunoglobulin heavy chain constant region is replaced with the nucleotide sequence of intron 1. In another aspect, the nucleotide sequence of intron 3 of the immunoglobulin heavy chain constant region is replaced with nucleotide sequence of intron 1.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequence of intron 3 of the immunoglobulin heavy chain constant region is replaced with a nucleotide sequence of an intron comprising about the same number of nucleotides as the nucleotide sequence of intron 1 of the immunoglobulin heavy chain constant region and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain. In one aspect, the nucleotide sequence of intron 1 in the immunoglobulin heavy chain constant region is deleted.

The present disclosure is also generally directed to a method of producing an immunoglobulin, comprising culturing a host cell in a medium under conditions in which the cell expresses the immunoglobulin; wherein the host cell comprises a nucleic acid comprising a nucleotide sequence encoding an immunoglobulin heavy chain, wherein the nucleotide sequence of intron 2 of the immunoglobulin heavy chain constant region is replaced with a nucleotide sequence of an intron comprising about the same number of nucleotides as the nucleotide sequence of intron 1 of the immunoglobulin heavy chain constant region. and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain. In one aspect, the nucleotide sequence of intron 1 in the immunoglobulin heavy chain constant region is deleted.

In one aspect, the expressed immunoglobulin has an IgG1, IgG2, IgG3, or IgG4 isotype. In another aspect, the expressed immunoglobulin is a human, humanized, chimeric, or resurfaced immunoglobulin.

In one aspect, the expressed immunoglobulin produced from a pool of clones has a harvest titer of at least 1,000 mg/L, of at least 1,500 mg/L, of at least 2,000 mg/L, of at least 2,500 mg/L, or of at least 3,000 mg/L. In another aspect, the expressed immunoglobulin produced from a top expressing clone has a harvest titer of at least 1,000 mg/L, of at least 1,500 mg/L, of at least 2,000 mg/L, of at least 3,000 mg/L, of at least 4,000 mg/L, of at least 5,000 mg/L, of at least 6,000 mg/L, of at least 7,000 mg/L, of at least 8,000 mg/L, of at least 9,000 mg/L, of at least 10,000 mg/L, of at least 11,000 mg/L, or of at least 12,000 mg/L.

In one aspect, the host cell is a eukaryotic cell. In another aspect, the eukaryotic cell is a CHO cell.

### BRIEF DESCRIPTION OF THE FIGURES

FIG. 1 shows a High Performance Size Exclusion Chromatograph of MAb1 and a depiction of the immunoglobulin variants produced.

FIGS. 2A-2B. FIG. 2A shows a diagram of the non-codon optimized genomic DNA (gDNA) and codon optimized complementary DNA (cDNA) for the immunoglobulin heavy chain constant region for MAb2. FIG. 2B shows the immunoglobulin titer (mg/L) on Day 14 of MAb2 production with either the gDNA or cDNA nucleotide sequences.

FIGS. 3A-3B. FIG. 3A shows a diagram of the non-codon optimized genomic DNA (gDNA) for the immunoglobulin heavy chain constant region, codon optimized complementary DNA (cDNA) for the immunoglobulin heavy chain constant region for MAb2, gDNA without intron 1 for the immunoglobulin heavy chain constant region for MAb2, gDNA without intron 2 for the immunoglobulin heavy chain constant region for MAb2, gDNA without intron 3 for the immunoglobulin heavy chain constant region for MAb2, and gDNA without any introns for the immunoglobulin heavy chain constant region for MAb2. FIG. 3B shows the immunoglobulin titer (mg/L) on Day 13 of MAb2 production using each of the following constructs: (1) non-codon optimized genomic DNA (gDNA) for the immunoglobulin heavy chain constant region; (2) codon optimized complementary DNA (cDNA) for the immunoglobulin heavy chain constant region for MAb2; (3) gDNA without intron 1 for the immunoglobulin heavy chain constant region for MAb2; (4) gDNA without intron 2 for the immunoglobulin heavy chain constant region for MAb2; (5) gDNA without intron 3 for the immunoglobulin heavy chain constant region for MAb2; and (6) gDNA without any introns for the immunoglobulin heavy chain constant region for MAb2.

FIGS. 4A-4D. FIG. 4A shows a graph depicting the average viable cell number (VCN) (×10⁶/mL) for each of the following constructs: (1) non-codon optimized genomic DNA (gDNA) for the immunoglobulin heavy chain constant region; (2) codon optimized complementary DNA (cDNA) for the immunoglobulin heavy chain constant region for MAb2; (3) gDNA without intron 1 for the immunoglobulin heavy chain constant region for MAb2; (4) gDNA without intron 2 for the immunoglobulin heavy chain constant region for MAb2; (5) gDNA without intron 3 for the immunoglobulin heavy chain constant region for MAb2; and (6) gDNA without any introns for the immunoglobulin heavy chain constant region for MAb2. FIG. 4B shows a graph depicting the cell viability (%) for each of the following constructs: (1) non-codon optimized genomic DNA (gDNA) for the immunoglobulin heavy chain constant region; (2) codon optimized complementary DNA (cDNA) for the immunoglobulin heavy chain constant region for MAb2; (3) gDNA without intron 1 for the immunoglobulin heavy chain constant region for MAb2; (4) gDNA without intron 2 for the immunoglobulin heavy chain constant region for MAb2; (5) gDNA without intron 3 for the immunoglobulin heavy chain constant region for MAb2; and (6) gDNA without any introns for the immunoglobulin heavy chain constant region for MAb2. FIG. 4C shows a graph depicting the integral of viable cells (IVC) (10⁹ cell day/L) for each of the following constructs: (1) non-codon optimized genomic DNA (gDNA) for the immunoglobulin heavy chain constant region; (2) codon optimized complementary DNA (cDNA) for the immunoglobulin heavy chain constant region for MAb2; (3) gDNA without intron 1 for the immunoglobulin heavy chain constant region for MAb2; (4) gDNA without intron 2 for the immunoglobulin heavy chain constant region for MAb2; (5) gDNA without intron 3 for the immunoglobulin heavy chain constant region for MAb2; and (6) gDNA without any introns for the immunoglobulin heavy chain constant region for MAb2. FIG. 4D shows a graph depicting the cell productivity (qP) (pg/(cell day)) for each of the following constructs: (1) non-codon optimized genomic DNA (gDNA) for the immunoglobulin heavy chain constant region; (2) codon optimized complementary DNA (cDNA) for the immunoglobulin heavy chain constant region for MAb2; (3) gDNA without intron 1 for the immunoglobulin heavy chain constant region for MAb2; (4) gDNA without intron 2 for the immunoglobulin heavy chain constant region for MAb2; (5) gDNA without intron 3 for the immunoglobulin heavy chain constant region for MAb2; and (6) gDNA without any introns for the immunoglobulin heavy chain constant region for MAb2.

FIGS. 5A-5D. FIG. 5A shows the immunoglobulin titer (mg/L) on Day 11 of MAb2 production with for each of the following constructs: (1) gDNA (non-codon optimized) with intron 2 removed from the immunoglobulin heavy chain constant region for MAb2; (2) gDNA (non-codon optimized) with introns 1 and 2 removed from the immunoglobulin heavy chain constant region for MAb2; (3) gDNA (non-codon optimized) with introns 2 and 3 removed from the immunoglobulin heavy chain constant region for MAb2; (4) gDNA (non-codon optimized) without any introns in the immunoglobulin heavy chain constant region for MAb2; (5) gDNA (codon optimized) with intron 2 removed from the immunoglobulin heavy chain constant region for MAb2; (6) gDNA (codon optimized) with introns 1 and 2 removed from the immunoglobulin heavy chain constant region for MAb2; (7) gDNA (codon optimized) with introns 2 and 3 removed from the immunoglobulin heavy chain constant region for MAb2; and (8) gDNA (codon optimized) without any introns in the immunoglobulin heavy chain constant region for MAb2. FIG. 5B shows a graph depicting the average viable cell number (VCN) (×10⁶/ml) for each of the following constructs: (1) gDNA (non-codon optimized) with intron 2 removed from the immunoglobulin heavy chain constant region for MAb2; (2) gDNA (non-codon optimized) with introns 1 and 2 removed from the immunoglobulin heavy chain constant region for MAb2; (3) gDNA (non-codon optimized) with introns 2 and 3 removed from the immunoglobulin heavy chain constant region for MAb2; (4) gDNA (non-codon optimized) without any introns in the immunoglobulin heavy chain constant region for MAb2; (5) gDNA (codon optimized) with intron 2 removed from the immunoglobulin heavy chain constant region for MAb2; (6) gDNA (codon optimized) with introns 1 and 2 removed from the immunoglobulin heavy chain constant region for MAb2; (7) gDNA (codon optimized) with introns 2 and 3 removed from the immunoglobulin heavy chain constant region for MAb2; (8) gDNA (codon optimized) without any introns in the immunoglobulin heavy chain constant region for MAb2; and (9) MAb3 (positive control). FIG. 5C shows a graph depicting the integral of viable cells (IVC) (10⁹ cell hr/L) for each of the each of the following constructs: (1) gDNA (non-codon optimized) with intron 2 removed from the immunoglobulin heavy chain constant region for MAb2; (2) gDNA (non-codon optimized) with introns 1 and 2 removed from the immunoglobulin heavy chain constant region for MAb2; (3) gDNA (non-codon optimized) with introns 2 and 3 removed from the immunoglobulin heavy chain constant region for MAb2; (4) gDNA (non-codon optimized) without any introns in the immunoglobulin heavy chain constant region for MAb2; (5) gDNA (codon optimized) with intron 2 removed from the immunoglobulin heavy chain constant region for MAb2; (6) gDNA (codon optimized) with introns 1 and 2 removed from the immunoglobulin heavy chain constant region for MAb2; (7) gDNA (codon optimized) with introns 2 and 3 removed from the immunoglobulin heavy chain constant region for MAb2; (8) gDNA (codon optimized) without any introns in the immunoglobulin heavy chain constant region for MAb2; and (9) MAb3 (positive control). FIG. 5D shows a graph depicting the cell productivity (qP) (pg/(cell day)) for each of the following constructs: (1) gDNA (non-codon optimized) with intron 2 removed from the immunoglobulin heavy chain constant region for MAb2; (2) gDNA (non-codon optimized) with introns 1 and 2 removed from the immunoglobulin heavy chain constant region for MAb2; (3) gDNA (non-codon optimized) with introns 2 and 3 removed from the immunoglobulin heavy chain constant region for MAb2; (4) gDNA (non-codon optimized) without any introns in the immunoglobulin heavy chain constant region for MAb2; (5) gDNA (codon optimized) with intron 2 removed from the immunoglobulin heavy chain constant region for MAb2; (6) gDNA (codon optimized) with introns 1 and 2 removed from the immunoglobulin heavy chain constant region for MAb2; (7) gDNA (codon optimized) with introns 2 and 3 removed from the immunoglobulin heavy chain constant region for MAb2; and (8) gDNA (codon optimized) without any introns in the immunoglobulin heavy chain constant region for MAb2.

FIGS. 6A-6B. FIG. 6A shows a diagram of the following constructs: (1) non-codon optimized genomic DNA (gDNA) from the immunoglobulin heavy chain constant region for MAb2; (2) non-codon optimized gDNA without intron 2 from the immunoglobulin heavy chain constant region for MAb2; (3) non-codon optimized gDNA without introns 2 and 3 from the immunoglobulin heavy chain constant region for MAb2; (4) non-codon optimized gDNA without introns 1 and 2 from the immunoglobulin heavy chain constant region for MAb2; (5) non-codon optimized gDNA with intron 3 at the position of intron 1 in the immunoglobulin heavy chain constant region for MAb2; (6) non-codon optimized gDNA with a modified intron 3 nucleotide sequence at the position of intron 1 in the immunoglobulin heavy chain constant region for MAb2; (7) non-codon optimized gDNA with intron 1 at the position of intron 3 in the immunoglobulin heavy chain constant region for MAb2; (8) non-codon optimized gDNA without any introns in the immunoglobulin heavy chain constant region for MAb2; (9) non-codon optimized genomic DNA (gDNA) with all introns and a wild type IgG1 for the immunoglobulin heavy chain constant region for MAb2; (10) non-codon optimized gDNA without introns 2 and 3 and a wild type IgG1 for the immunoglobulin heavy chain constant region for MAb2; (11) non-codon optimized gDNA without introns 1 and 2 and a wild type IgG1 for the immunoglobulin heavy chain constant region for MAb2; and (12) non-codon optimized gDNA and a wild type IgG1 for the immunoglobulin heavy chain constant region for MAb2. FIG. 6B shows the immunoglobulin titer (mg/L) on Day 11 of MAb2 production with the following constructs: (1) non-codon optimized genomic DNA (gDNA) from the immunoglobulin heavy chain constant region for MAb2; (2) non-codon optimized gDNA without intron 2 from the immunoglobulin heavy chain constant region for MAb2; (3) non-codon optimized gDNA without introns 2 and 3 from the immunoglobulin heavy chain constant region for MAb2; (4) non-codon optimized gDNA without introns 1 and 2 from the immunoglobulin heavy chain constant region for MAb2; (5) non-codon optimized gDNA with intron 3 at the position of intron 1 in the immunoglobulin heavy chain constant region for MAb2; (6) non-codon optimized gDNA with a modified intron 3 nucleotide sequence at the position of intron 1 in the immunoglobulin heavy chain constant region for MAb2; (7) non-codon optimized gDNA with intron 1 at the position of intron 3 in the immunoglobulin heavy chain constant region for MAb2; (8) non-codon optimized gDNA without any introns in the immunoglobulin heavy chain constant region for MAb2; (9) non-codon optimized genomic DNA (gDNA) with all introns and a wild type IgG1 for the immunoglobulin heavy chain constant region for MAb2; (10) non-codon optimized gDNA without introns 2 and 3 and a wild type IgG1 for the immunoglobulin heavy chain constant region for MAb2; (11) non-codon optimized gDNA without introns 1 and 2 and a wild type IgG1 for the immunoglobulin heavy chain constant region for MAb2; and (12) non-codon optimized gDNA and a wild type IgG1 for the immunoglobulin heavy chain constant region for MAb2.

FIG. 7 shows a diagram of the non-codon optimized genomic DNA (gDNA) for the immunoglobulin heavy chain constant region for MAb2, MAb1, MAb3, and MAb4; non-codon optimized gDNA without introns 2 and 3 for the immunoglobulin heavy chain constant region for MAb2, MAb1, MAb3, and MAb4; and non-codon optimized gDNA without any introns for the immunoglobulin heavy chain constant region for MAb2, MAb1, MAb3, and MAb4.

FIG. 8 shows the immunoglobulin titer (mg/L) on Day 11 of non-codon optimized genomic DNA (gDNA) for the immunoglobulin heavy chain constant region for MAb2, MAb1, MAb3, and MAb4; non-codon optimized gDNA without introns 2 and 3 for the immunoglobulin heavy chain constant region for MAb2, MAb1, MAb3, and MAb4; and non-codon optimized gDNA without any introns for the immunoglobulin heavy chain constant region for MAb2, MAb1, MAb3, and MAb4.

FIG. 9 shows a graph depicting the change in titer levels over time of non-codon optimized genomic DNA (gDNA) for the immunoglobulin heavy chain constant region for MAb2, MAb1, MAb3, and MAb4; non-codon optimized gDNA without introns 2 and 3 for the immunoglobulin heavy chain constant region for MAb2, MAb1, MAb3, and MAb4; and non-codon optimized gDNA without any introns for the immunoglobulin heavy chain constant region for MAb2, MAb1, MAb3, and MAb4.

FIG. 10 shows a diagram of the genomic DNA arrangement for an immunoglobulin heavy chain constant region.

### DETAILED DESCRIPTION

To facilitate an understanding of the present disclosure, a number of terms and phrases are defined below.

### I. DEFINITIONS

As used herein, the terms “immunoglobulin” “antibody” and “antibodies” are terms of art and can be used interchangeably herein and refer to a molecule or a complex of molecules with at least one antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), bispecific antibodies, and multi-specific antibodies. In certain aspects, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain aspects, antibodies described herein are IgG antibodies, or a class (e.g., human IgG₁, IgG₂, or IgG₄) or subclass thereof. In a specific aspect, the antibody is a humanized monoclonal antibody. In another specific aspect, the antibody is a human monoclonal antibody, e.g., that is an immunoglobulin. In certain aspects, an antibody described herein is an IgG₁, IgG₂, or IgG₄ antibody.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the complementarity determining regions (CDR)). The antigen-binding region can be derived from any animal species, such as rodents (e.g., mouse, rat, or hamster) and humans.

A “monoclonal” antibody refers to a homogeneous antibody population involved in the highly specific recognition and binding of a single antigenic determinant, or epitope. This is in contrast to polyclonal antibodies that typically include different antibodies directed against different antigenic determinants. The term “monoclonal” antibody encompasses both intact and full-length immunoglobulin molecules as well Fab, Fab′, F(ab′)2, Fv), single chain (scFv), fusion proteins comprising an antibody portion, and any other modified immunoglobulin molecule comprising an antigen recognition site. Furthermore, a “monoclonal” antibody refers to such antibodies made in any number of manners including but not limited to by hybridoma, phage selection, recombinant expression, and transgenic animals.

The term “chimeric” antibodies refers to antibodies wherein the amino acid sequence is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in antibodies derived from another (usually human) to avoid eliciting an immune response in that species.

The term “humanized” antibody refers to forms of non-human (e.g. murine) antibodies that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody thereof can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996).

The term “resurfaced antibody” or “resurfaced antibodies” means an murine antibody that is redesigned to resemble human antibodies by humanizing only those amino acids that are accessible at the surface of the V-regions of the recombinant FV. The resurfacing of murine monoclonal antibodies to reduce their immunogenicity could be beneficial in maintaining the avidity of the original monoclonal antibody in the reshaped version, because the natural framework-CDR interactions are retained.

The term “human antibody” means an antibody having an amino acid sequence derived from a human immunoglobulin gene locus, where such antibody is made using any technique known in the art.

The variable region typically refers to a portion of an antibody, generally, a portion of a light or heavy chain, typically about the amino-terminal 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain aspects, the variable region is a human variable region. In certain aspects, the variable region comprises rodent or murine CDRs and human framework regions (FRs). In particular aspects, the variable region is a primate (e.g., non-human primate) variable region. In certain aspects, the variable region comprises rodent or murine CDRs and primate (e.g., non-human primate) framework regions (FRs).

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. An immunoglobulin “constant region” or “constant domain” can contain a CH1 domain, a hinge, a CH2 domain, and a CH3 domain or a subset of these domains, e.g., a CH2 domain and a CH3 domain. In certain aspects provided herein, an immunoglobulin constant region does not contain a CH1 domain. In certain aspects provided herein, an immunoglobulin constant region does not contain a hinge. In certain aspects provided herein, an immunoglobulin constant region contains a CH2 domain and a CH3 domain.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” and refers to the fragment of an antibody that will readily form a protein crystal. Distinct protein fragments, which were originally described by proteolytic digestion, can define the overall general structure of an immunoglobulin protein. An “Fc region” or “Fc domain” contains a CH2 domain, a CH3 domain, and optionally all or a portion of a hinge. An “Fc region” or “Fc domain” can refer to a single polypeptide or to two disulfide-linked polypeptides. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of a naturally occurring antibody. In certain aspects, a wild type immunoglobulin hinge region sequence is human, and can comprise a human IgG hinge region. An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in US 2013/0129723 and US 2013/0095097.

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant regions. Light chain amino acid sequences are well known in the art. In specific aspects, the light chain is a human light chain.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain aspects, the polypeptides can occur as single chains or associated chains.

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “intron” as used herein refers to a sequence of nucleotides that is transcribed into RNA and is then typically removed from the RNA by splicing to create a mature form of an RNA, for example, an mRNA. Typically, nucleotide sequences of introns are not incorporated into mature RNAs, nor are intron sequences or a portion thereof typically translated and incorporated into a polypeptide. Splice signal sequences such as splice donors and acceptors are used by the splicing machinery of a cell to remove introns from RNA.

The term “vector” as used herein refers to a nucleic acid, linear or circular, that comprises a segment according to the nucleic acid of interest.

The term “expression vector,” as used herein refers to a nucleic acid molecule, linear or circular, comprising one or more expression units. In addition to one or more expression units, an expression vector can also include additional nucleic acid segments such as, Expression vectors are generally derived from plasmid or viral DNA, or can contain elements of both.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “viable cell number” as used herein refers to the number of viable (living) cells present in a culture.

The term “cell viability” as used herein refers to the ability of cells in culture to survive under a given set of culture conditions or experimental variations. The term as used herein also refers to that portion of cells, which are alive at a particular time in relation to the total number of cells, living and dead, in the culture at that time.

The term “harvest titer” or “titer,” as used herein refers to the total amount of expressed polypeptide or immunoglobulin produced in a cell culture divided by a given amount of medium volume.

The term “qP” as used herein refers to cell-specific productivity and is determined from the total immunoglobulin produced divided by the integral of viable cells.

The term “IVC” as used herein refers to integral of viable cells and is calculated by

∫₀^{t}XVdt

where X is the viable cell concentration, V is the volume of the culture, and t is time.

A polypeptide, antibody, nucleic acid, vector, cell, or composition which is “isolated” is a polypeptide, antibody, nucleic acid, vector, cell, or composition which is in a form not found in nature. Isolated polypeptides, antibodies, nucleic acids, vectors, cell or compositions include those which have been purified to a degree that they are no longer in a form in which they are found in nature. In some aspects, an antibody, nucleic acid, vector, cell, or composition which is isolated is substantially pure. As used herein, “substantially pure” refers to material which is at least 50% pure (i.e., free from contaminants). In some instances, a material is at least 90% pure, at least 95% pure, at least 98% pure, or at least 99% pure.

It should be understood that the terms “a” and “an” as used herein refer to “one or more” of the enumerated components unless otherwise indicated.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided and part of the present application's disclosure. In this disclosure, “comprises,” “comprising,” “containing” and “having” and the like can have the meaning ascribed to them in U.S. and European Patent law and can mean “includes,” “including,” and the like; “consisting essentially of” or “consists essentially” likewise has the meaning ascribed in U.S. and European Patent law. It should be appreciated that as far as U.S. Patent law is concerned, the term is open-ended, allowing for the presence of more than that which is recited so long as basic or novel characteristics of that which is recited is not changed by the presence of more than that which is recited, but excludes prior art aspects. It should also be appreciated that as far as European Patent law is concerned the use of “consisting essentially of” or “comprising substantially” means that specific further components can be present, namely those not materially affecting the essential characteristics of the compound or composition.

As used herein, the terms “about” and “approximately,” when used to modify a numeric value or numeric range, indicate that deviations of up to 5% above or 5% below the value or range remain within the intended meaning of the recited value or range.

### II. NUCLEIC ACIDS ENCODING IMMUNOGLOBULINS

Human immunoglobulin G (IgG) contains a heavy chain polypeptide and a light chain polypeptide, which together form an immunoglobulin. The immunoglobulin light chain has a variable light chain, which comprises the variable light region complementarity determining regions (CDRs) that help bind to an epitope. The immunoglobulin light chain also contains a light chain constant region. The IgG light chain can be either a kappa or lambda light chain.

The immunoglobulin heavy chain has a variable heavy chain, which comprises the variable heavy region complementarity determining regions (CDRs) that help bind to an epitope. The immunoglobulin heavy chain also contains a heavy chain constant region. In the IgG heavy chain constant region, there is a three constant domains (CH1, CH2, and CH3) and a hinge region.

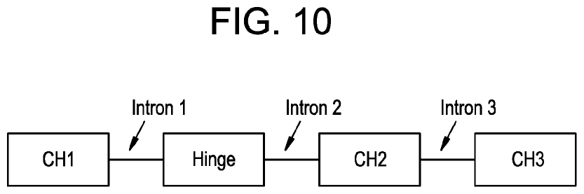

The nucleotide sequence that encodes the human IgG heavy chain constant region contains three introns (see, FIG. 10). Intron 1 in the human IgG heavy chain constant region is located between the exons that encode the CH1 and hinge regions. Intron 2 in the human IgG heavy chain constant region is located between the exons that encode the hinge and CH2 regions. Intron 3 in the human IgG heavy chain constant region is located between the exons that encode the CH2 and CH3 regions.

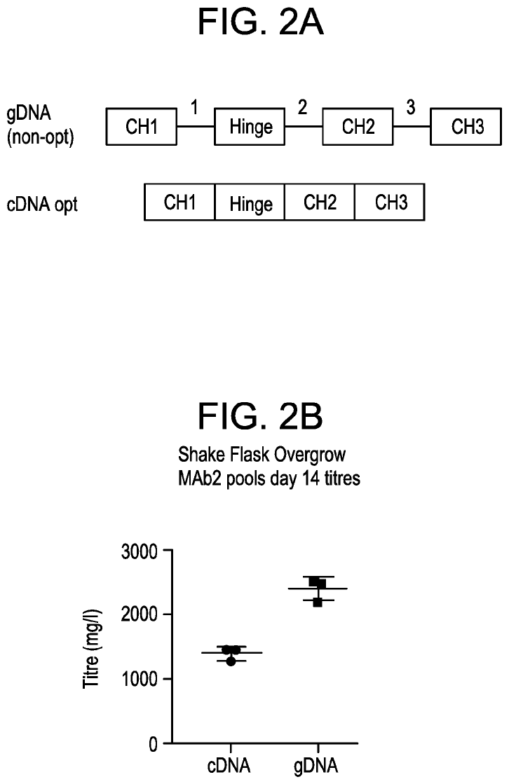

It is known that introns are linked to mRNA export out of the nucleus through the nuclear pore complexes. See generally Kohler A. et al., Nature Review Molecular Cell Biology 8:761-773 (2007); Bjork, P. et al., Seminars in Cell & Developmental Biology 32:47-54 (2014); Reed, R. Current Opinion in Cell Biology, 15:326-331 (2003). Thus, when preparing nucleic acids for recombinant immunoglobulin production, the endogenous introns are usually not excised as nucleic acids with introns typically increase immunoglobulin production titer as compared to the corresponding cDNA version. See, e.g., FIG. 2.

Although immunoglobulin production with nucleic acids containing endogenous introns in the human IgG heavy chain leads to increased immunoglobulin titer, the introns can introduce incorrect splice sites that can lead to immunoglobulin fragments or variants, thus reducing product purity. Introducing immunoglobulin fragments or variants in the immunoglobulin pool can increase difficulty in purifying these fragments and variants, thus exerting undue burden on purification processes and contributing to lower product purity.

In one aspect, deletion of the nucleotide sequence for one or two introns in the IgG heavy chain constant region leads to decreased production of immunoglobulin fragments or variants, and increased immunoglobulin titer when expressed with the appropriate nucleic acid encoding a IgG light chain.

In certain aspects, the disclosure encompasses a nucleic acid comprising a nucleotide sequence encoding an immunoglobulin heavy chain, wherein the nucleotide sequences of the second and third introns of the immunoglobulin heavy chain constant region are deleted. In certain aspects, the disclosure encompasses a nucleic acid comprising a nucleotide sequence encoding an immunoglobulin heavy chain, wherein the nucleotide sequence comprises the sequence of the first intron and the sequences of the second and third introns of the immunoglobulin heavy chain constant region are deleted. In certain aspects, the disclosure encompasses a nucleic acid comprising a nucleotide sequence encoding an immunoglobulin heavy chain, wherein the nucleotide sequence only comprises the first intron of the immunoglobulin heavy chain constant region. In certain aspects, the disclosure encompasses a nucleic acid comprising a nucleotide sequence encoding an immunoglobulin heavy chain, wherein the nucleotide sequence only comprises the second intron of the immunoglobulin heavy chain constant region. In certain aspects, the disclosure encompasses a nucleic acid comprising a nucleotide sequence encoding an immunoglobulin heavy chain, wherein the nucleotide sequence only comprises the third intron of the immunoglobulin heavy chain constant region.

In certain aspects, the disclosure encompasses a nucleic acid comprising a nucleotide sequence encoding an immunoglobulin heavy chain, wherein the nucleotide sequences of one intron of three endogenous introns of the immunoglobulin heavy chain constant region are deleted. In some instances, the first intron is deleted. In some instances, the second intron is deleted. In some instances, the third intron is deleted. In certain aspects, the disclosure encompasses a nucleic acid comprising a nucleotide sequence encoding an immunoglobulin heavy chain, wherein the nucleotide sequence comprises the sequence of the second and third introns, but the sequence of the first intron of the immunoglobulin heavy chain constant region is deleted. In certain aspects, the disclosure encompasses a nucleic acid comprising a nucleotide sequence encoding an immunoglobulin heavy chain, wherein the nucleotide sequence comprises the sequence of the first and third introns, but the sequence of the second intron of the immunoglobulin heavy chain constant region is deleted. In certain aspects, the disclosure encompasses a nucleic acid comprising a nucleotide sequence encoding an immunoglobulin heavy chain, wherein the nucleotide sequence comprises the sequence of the first and second introns, but the sequence of the third intron of the immunoglobulin heavy chain constant region is deleted.

In certain aspects, the disclosure encompasses a nucleic acid comprising a nucleotide sequence encoding an immunoglobulin heavy chain, wherein the nucleotide sequences of two introns of the three endogenous introns in the immunoglobulin heavy chain constant region are deleted. In some instances, the first and second introns are deleted. In some instances, the first and third introns are deleted. In some instances, the second and third introns are deleted. In certain aspects, the disclosure encompasses a nucleic acid comprising a nucleotide sequence encoding an immunoglobulin heavy chain, wherein the nucleotide sequence comprises the sequence of the first intron, but the sequence of the second and third introns of the immunoglobulin heavy chain constant region are deleted. In certain aspects, the disclosure encompasses a nucleic acid comprising a nucleotide sequence encoding an immunoglobulin heavy chain, wherein the nucleotide sequence comprises the sequence of the second intron, but the sequence of the first and third introns of the immunoglobulin heavy chain constant region are deleted. In certain aspects, the disclosure encompasses a nucleic acid comprising a nucleotide sequence encoding an immunoglobulin heavy chain, wherein the nucleotide sequence comprises the sequence of the third intron, but the sequence of the first and second introns of the immunoglobulin heavy chain constant region are deleted.

In certain aspects, the disclosure encompasses a nucleic acid comprising a nucleotide sequence encoding an immunoglobulin heavy chain, wherein the nucleotide sequence of the first intron is deleted and the nucleotide sequence of the second and/or third introns of the immunoglobulin heavy chain constant region are deleted, and wherein the nucleotide sequences of the second and/or third introns are substituted with the nucleotide sequence of the first intron. In some instances, the nucleotide sequence of the second intron is substituted with the nucleotide sequence of the first intron. In some instances, the nucleotide sequence of the third intron is substituted with the nucleotide sequence of the first intron. In certain aspects, the disclosure encompasses a nucleic acid comprising a nucleotide sequence encoding an immunoglobulin heavy chain, wherein the nucleotide sequences of the first and second introns are deleted from the immunoglobulin heavy chain constant region, and wherein the nucleotide sequence of the second intron is substituted with the nucleotide sequence of the first intron. In certain aspects, the disclosure encompasses a nucleic acid comprising a nucleotide sequence encoding an immunoglobulin heavy chain, wherein the nucleotide sequences of the first and third introns are deleted from the immunoglobulin heavy chain constant region, and wherein the nucleotide sequence of the third intron is substituted with the nucleotide sequence of the first intron. In certain aspects, the disclosure encompasses a nucleic acid comprising a nucleotide sequence encoding an immunoglobulin heavy chain, wherein the nucleotide sequence of the first intron is deleted and the nucleotide sequences of the second and third introns of the immunoglobulin heavy chain constant region are deleted, and wherein the nucleotide sequences of the second and third introns are each substituted with the nucleotide sequence of the first intron.

In certain aspects, the disclosure encompasses a nucleic acid comprising a nucleotide sequence encoding an immunoglobulin heavy chain, wherein the nucleotide sequences of the second and/or third introns in the immunoglobulin heavy chain constant region are substituted with the nucleotide sequence of the first intron. In some instances, the nucleotide sequence of the second intron is substituted with the nucleotide sequence of the first intron. In some instances, the nucleotide sequence of the third intron is substituted with the nucleotide sequence of the first intron. In certain aspects, the disclosure encompasses a nucleic acid comprising a nucleotide sequence encoding an immunoglobulin heavy chain, wherein the nucleotide sequence of the second intron in the immunoglobulin heavy chain constant region is substituted with the nucleotide sequence of the first intron without deleting the sequences of the first and third introns. In certain aspects, the disclosure encompasses a nucleic acid comprising a nucleotide sequence encoding an immunoglobulin heavy chain, wherein the nucleotide sequence of the third intron in the immunoglobulin heavy chain constant region is substituted with the nucleotide sequence of the first intron without deleting the sequences of the first and second introns. In certain aspects, the disclosure encompasses a nucleic acid comprising a nucleotide sequence encoding an immunoglobulin heavy chain, wherein the nucleotide sequences of the second and third introns in the immunoglobulin heavy chain constant region are substituted with the nucleotide sequence of the first intron without deleting the sequence of the first intron.

In certain aspects, the disclosure encompasses a nucleic acid comprising a nucleotide sequence encoding an immunoglobulin heavy chain, wherein the nucleotide sequence of the third intron in the immunoglobulin heavy chain constant region is replaced with a nucleotide sequence of an intron comprising about the same number of nucleotides as the nucleotide sequence of the first intron of the immunoglobulin heavy chain constant region. In some instances, the nucleotide sequence of the first intron in the immunoglobulin heavy chain constant region is deleted. In certain aspects, the disclosure encompasses a nucleic acid comprising a nucleotide sequence encoding an immunoglobulin heavy chain, wherein the nucleotide sequence of the third intron in the immunoglobulin heavy chain constant region is replaced with a nucleotide sequence of an intron comprising about the same number of nucleotides as the nucleotide sequence of the first intron of the immunoglobulin heavy chain constant region and the sequences of the first and/or second introns are deleted.

In certain aspects, the disclosure encompasses a nucleic acid comprising a nucleotide sequence encoding an immunoglobulin heavy chain, wherein the nucleotide sequence of the second intron in the immunoglobulin heavy chain constant region is replaced with a nucleotide sequence of an intron comprising about the same number of nucleotides as the nucleotide sequence of the first intron of the immunoglobulin heavy chain constant region. In some instances, the nucleotide sequence of the first intron in the immunoglobulin heavy chain constant region is deleted. In certain aspects, the disclosure encompasses a nucleic acid comprising a nucleotide sequence encoding an immunoglobulin heavy chain, wherein the nucleotide sequence of the second intron in the immunoglobulin heavy chain constant region is replaced with a nucleotide sequence of an intron comprising about the same number of nucleotides as the nucleotide sequence of the first intron of the immunoglobulin heavy chain constant region and the sequences of the first and/or third introns are deleted.

In any of the above aspects, the nucleic acid comprising a nucleotide sequence encoding an immunoglobulin heavy chain can also be expressed with a nucleic acid encoding an immunoglobulin light chain. In some instances, the immunoglobulin light chain is a kappa light chain. In some instances, the immunoglobulin light chain is a lambda light chain.

In any of the above aspects, the nucleic acid expresses an immunoglobulin at a higher titer than a nucleic acid containing all intron sequences in the heavy chain constant region when expressed with a nucleic acid encoding an immunoglobulin light chain.

In any of the above aspects, the nucleic acid expresses an immunoglobulin at a higher titer than a nucleic acid containing no intron sequences in the heavy chain constant region when expressed with a nucleic acid encoding an immunoglobulin light chain.

In any of the above aspects, the nucleic acid is not codon optimized. In any of the above aspects, the nucleic acid is codon optimized.

In any of the above aspects, the immunoglobulin expressed has an IgG₁, IgG₂, IgG₃, or IgG₄ isotype.

In any of the above aspects, the immunoglobulin expressed is a human, humanized, chimeric, or resurfaced immunoglobulin.

The nucleic acids of the disclosure can be in the form of RNA or in the form of DNA. DNA includes genomic DNA or synthetic DNA; and can be double-stranded or single-stranded, and if single stranded can be the coding strand or non-coding (anti-sense) strand. In some aspects, the nucleic acid is a DNA lacking one more endogenous introns.

In some aspects, a nucleic acid comprises a non-naturally occurring nucleotide. In some aspects, a nucleic acid is recombinantly produced.

In certain aspects, the nucleic acid is isolated.

### III. CELLS AND VECTORS

Vectors and cells comprising the nucleic acids described herein are also provided.

In certain aspects, provided herein are cells (e.g., host cells) that comprise expression vectors that express (e.g., recombinantly) the nucleic acids described herein that encode an immunoglobulin. Provided herein are vectors (e.g., expression vectors) comprising nucleotide sequences encoding immunoglobulin heavy and light chains for recombinant expression in host cells, e.g., mammalian host cells. Also provided herein are host cells comprising such vectors for recombinantly expressing nucleic acids that comprise a nucleotide sequence that encodes an immunoglobulin.

Recombinant expression of an immunoglobulin involves an expression vector containing a nucleic acid comprising a nucleotide sequence encoding an immunoglobulin described herein (e.g., an IgG1, an IgG2, an IgG3, or an IgG4). In some aspects, the vector comprises a nucleic acid as described herein that contains a nucleotide sequence that encodes an immunoglobulin. In some aspects, a host cell comprises the vector. The present disclosure also provides constructs in the form of plasmids, vectors, transcription or expression cassettes which comprise a nucleic acid as described herein. In some aspects, the vector is an expression vector that additional nucleotide sequences (e.g., a promoter) to help recombinant immunoglobulin production in host cells.

An expression vector can be transferred to a cell (e.g., host cell) by conventional techniques and the resulting cells can then be cultured by conventional techniques to produce an immunoglobulin described herein. Thus, provided herein are host cells containing a polynucleotide encoding an immunoglobulin or a polypeptide thereof described herein operably linked to a promoter for expression of such sequences in the host cell.

In certain aspects, a host cell contains a vector comprising nucleic acids encoding the immunoglobulin heavy and light chain polypeptides of an immunoglobulin described herein. In certain aspects, a host cell contains multiple different vectors comprising the nucleic acids encoding all of the polypeptides of an immunoglobulin described herein.

A vector or combination of vectors can comprise nucleic acids encoding two or more polypeptides that interact to form an immunoglobulin described herein: e.g., a first nucleic acid encoding a heavy chain and a second nucleic acid encoding a light chain. Where the two polypeptides are encoded by nucleic acids in two separate vectors, the vectors can be transfected into the same host cell.

A variety of host-expression vector systems can be utilized to express immunoglobulins or polypeptides thereof (e.g., an immunoglobulin constant region; a heavy or light chain) described herein. Such host-expression systems represent vehicles by which the coding sequences of interest can be produced and subsequently purified, but also represent cells which can, when transformed or transfected with the appropriate nucleotide coding sequences, express an immunoglobulin or polypeptide thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Once an immunoglobulin or a polypeptide thereof (e.g., an immunoglobulin constant region; a heavy or light chain) described herein has been produced by recombinant expression, it can be purified by any method known in the art for purification of an antibody, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein (e.g., a FLAG tag, a his tag, or avidin) or otherwise known in the art to facilitate purification.

### IV. IMMUNOGLOBULIN PRODUCTION

Immunoglobulins can be produced by any method known in the art for the synthesis of immunoglobulins, for example, by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, isolated nucleic acids having a nucleotide sequence encoding any of the immunoglobulin heavy chain constant regions, and optionally isolated nucleic acids having a nucleotide sequence encoding the immunoglobulin heavy chain variable regions and/or the immunoglobulin light chain of the present disclosure are provided. Such nucleic acids may encode an amino acid sequence comprising the CL and/or an amino acid sequence comprising the CH of an immunoglobulin (e.g., the light and/or heavy constants chains of the immunoglobulin). Such nucleic acids may encode an amino acid sequence comprising the V_{L} and/or an amino acid sequence comprising the V_{H} of an immunoglobulin (e.g., the light and/or heavy variable chains of the immunoglobulin). In some aspects, one or more vectors (e.g., expression vectors) comprising such nucleic acids are provided. In some aspects, a host cell comprising such nucleic acid is also provided. In some aspects, the host cell comprises (e.g., has been transfected with): (1) a vector comprising a nucleic acid that encodes an amino acid sequence comprising the light chain of the immunoglobulin and an amino acid sequence comprising the heavy chain of the immunoglobulin, or (2) a first vector comprising a nucleic acid that encodes an amino acid sequence comprising the light chain of the immunoglobulin and a second vector comprising a nucleic acid that encodes an amino acid sequence comprising the heavy chain of the immunoglobulin.

In some aspects, a host cell comprising a nucleic acid of the present disclosure encoding an immunoglobulin is cultured under conditions suitable for expression of the antibody. For recombinant production of an immunoglobulin using nucleic acids of the present disclosure, one or more nucleic acids encoding the heavy and/or light chain is isolated and inserted into one or more vectors for further cloning and/or expression in a host cell as described herein.

In a certain aspect, the host cell comprises a nucleic acid comprising a nucleotide sequence encoding an immunoglobulin heavy chain, wherein the nucleotide sequences of the second and third introns of the immunoglobulin heavy chain constant region are deleted and a nucleic acid encoding an immunoglobulin light chain, wherein the host cell expresses the immunoglobulin at a higher titer than host cell comprising a nucleic acid encoding an immunoglobulin heavy chain constant region comprising all intron sequences or no intron sequences. In a particular aspect, the cell is an isolated cell. In some aspects, the nucleic sequence of the first intron is deleted. In some aspects, the nucleic sequence of the second intron is deleted. In some aspects, the nucleic sequence of the third intron is deleted. In some aspects, the nucleotide sequences of the first and second introns are deleted. In some aspects, the nucleotide sequences of the first and third introns are deleted. In some aspects, the nucleotide sequence of the second intron is substituted with the nucleotide sequence of the first intron. In some aspects, the nucleotide sequence of the third intron is substituted with the nucleotide sequence of the first intron. In some aspects, the nucleotide sequence of the second intron is replaced with the nucleotide sequence of the first intron. In some aspects, the nucleotide sequence of the third intron is replaced with the nucleotide sequence of the first intron. In a certain aspect, the nucleotide sequence of the third intron in the immunoglobulin heavy chain constant region is replaced with a nucleotide sequence of an intron comprising about the same number of nucleotides as the nucleotide sequence of the first intron of the immunoglobulin heavy chain constant region. In a certain aspect, the nucleotide sequence of the second intron in the immunoglobulin heavy chain constant region is replaced with a nucleotide sequence of an intron comprising about the same number of nucleotides as the nucleotide sequence of the first intron of the immunoglobulin heavy chain constant region. In some aspects, the nucleotide sequence of the first intron in the immunoglobulin heavy chain constant region is also deleted.

In any of the above aspects, the immunoglobulin light chain is a kappa light chain or a lambda light chain.

In any of the above aspects, the nucleic acid is codon optimized. In any of the above aspects, the nucleic acid is not codon optimized.

In any of the above aspects, the immunoglobulin has an IgG1, IgG2, IgG3, or IgG4 isotype.

In any of the above aspects, the immunoglobulin is a human, humanized, chimeric, or resurfaced immunoglobulin.

In any of the above aspects, the immunoglobulin produced from a pool of clones has a harvest titer of at least 1,000 mg/L. In any of the above aspects, the immunoglobulin produced from a pool of clones has a harvest titer of at least 1,500 mg/L. In any of the above aspects, the immunoglobulin produced from a pool of clones has a harvest titer of at least 2,000 mg/L. In any of the above aspects, the immunoglobulin produced from a pool of clones has a harvest titer of at least 2,500 mg/L. In any of the above aspects, the immunoglobulin produced from a pool of clones has a harvest titer of at least 3,000 mg/L.

In any of the above aspects, the immunoglobulin produced from a top expressing clone has a harvest titer of at least 1,000 mg/L. In any of the above aspects, the immunoglobulin produced from a top expressing clone has a harvest titer of at least 1,500 mg/L. In any of the above aspects, the immunoglobulin produced from a top expressing clone has a harvest titer of at least 2,000 mg/L. In any of the above aspects, the immunoglobulin produced from a top expressing clone has a harvest titer of at least 3,000 mg/L. In any of the above aspects, the immunoglobulin produced from a top expressing clone has a harvest titer of at least 4,000 mg/L. In any of the above aspects, the immunoglobulin produced from a top expressing clone has a harvest titer of at least 5,000 mg/L. In any of the above aspects, the immunoglobulin produced from a top expressing clone has a harvest titer of at least 6,000 mg/L. In any of the above aspects, the immunoglobulin produced from a top expressing clone has a harvest titer of at least 7,000 mg/L. In any of the above aspects, the immunoglobulin produced from a top expressing clone has a harvest titer of at least 8,000 mg/L. In any of the above aspects, the immunoglobulin produced from a top expressing clone has a harvest titer of at least 9,000 mg/L. In any of the above aspects, the immunoglobulin produced from a top expressing clone has a harvest titer of at least 10,000 mg/L. In any of the above aspects, the immunoglobulin produced from a top expressing clone has a harvest titer of at least 11,000 mg/L. In any of the above aspects, the immunoglobulin produced from a top expressing clone has a harvest titer of at least 12,000 mg/L.

In any of the above aspects, the host cell is a eukaryotic cell. In any of the above aspects, the eukaryotic cell is a CHO cell.

In some aspects, the exogenous nucleic acids have been introduced into the cell.

In some aspects, the method further comprises the step of purifying the immunoglobulin from the cell or host cell.

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

It is understood that the examples and aspects described herein are for illustrative purposes only and that various modifications or changes in light thereof will be suggested to persons skilled in the art and are to be included within the spirit and purview of this application.

### Example 1. Characterizing Mis-Spliced Immunoglobulin Variants

Introns are important in several areas, including regulating alternative splicing, enhancing gene expression, and controlling mRNA transport from the nucleus. Thus, nucleic acids used in expression vectors to produce immunoglobulins containing the endogenous introns. However, this can cause mis-spliced immunoglobulin variants that can be difficult to purify and/or reduce harvest titer of immunoglobulin product.

Cell culture production of MAb1 resulted in three immunoglobulin variants due to intron splice variants. The expression vector containing the light chain and heavy chain sequences for each immunoglobulin, including the endogenous intron sequences in the heavy chain constant region, were linearized and transfected into Chinese Hamster Ovary (CHO) cells. Transfection of the linearized expression vector was by nucleofection to generate a pool of cells. The linearized expression vector intergrated into the CHO genome via random intergration. The pool of cells expressing MAb1 was cloned out to generate the clonal manufacturing cell line using the appropriate cell line development process at the time.

CHO cells were grown and induced to begin production of MAb1. Immunoglobulins were harvested, and the immunoglobulins were purified using high performance size exclusion chromatography (HPSEC). For HPSEC fractionation, MAb1 was injected onto a TSK-gel G3000SWxL column (7.8 mm×30 cm; Tosoh Bioscience, King of Prussia, PA, USA) at ambient column temperature. The sample was eluted isocratically with a mobile phase composed of 0.1 M sodium phosphate, 0.1 M sodium sulfate, pH 6.8 at a flow rate of 1.0 mL/min. Fractions collected from multiple injections were pooled and concentrated prior to characterization and analysis. See Harris, C. et al., MABS, 11:1452-1463 (2019).

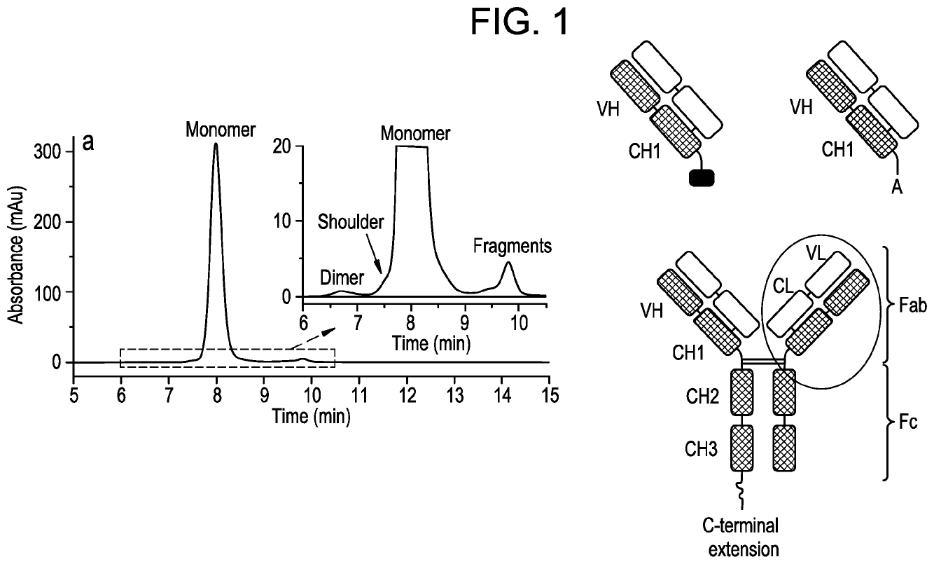

As shown in FIG. 1, HPSEC reveals 3 splice variants: a monomer immunoglobulin with an extension and two fragments. The immunoglobulin variant with the extension was identified to have an extra lambda light chain constant domain on the C-terminus of the immunoglobulin. This was caused by an alternative heavy chain transcript that had an extra lambda light chain constant domain on the C-terminus of the heavy chain. The two fragments were determined to be splice variants related to intron 2, in between the hinge and CH2 region. Specifically, one fragment is caused by an in-frame stop codon resulting in a truncated heavy chain, and the other is a mis-splicing event that results in a frame shift creating a stop codon and a truncated heavy chain. Thus, the immunoglobulin fragment variants are produced by the intron in the immunoglobulin heavy chain constant region.

### Example 2. Engineering Nucleic Acids to Reduce Immunoglobulin Splice Variants

To eliminate the immunoglobulin fragments produced in Example 1, a codon-optimized cDNA nucleotide sequence was created without any introns in the immunoglobulin heavy chain constant region for MAb2. The cDNA version removed intron 2 in the immunoglobulin heavy chain constant region, preventing production of the two fragments.

However, it is well known that introns are needed to enhance protein expression in CHO cells. Thus, CHO cells were transfected with the non-codon optimized gDNA or codon optimized cDNA by nucleofection to generate pools, grown, and induced to produce the immunoglobulins as in a pool fed-batch process in shake flasks. See FIG. 2A. FIG. 2B demonstrates that the cDNA version of MAb2 produces significantly less immunoglobulin, as shown by harvest titer, compared to the genomic DNA (gDNA) version.

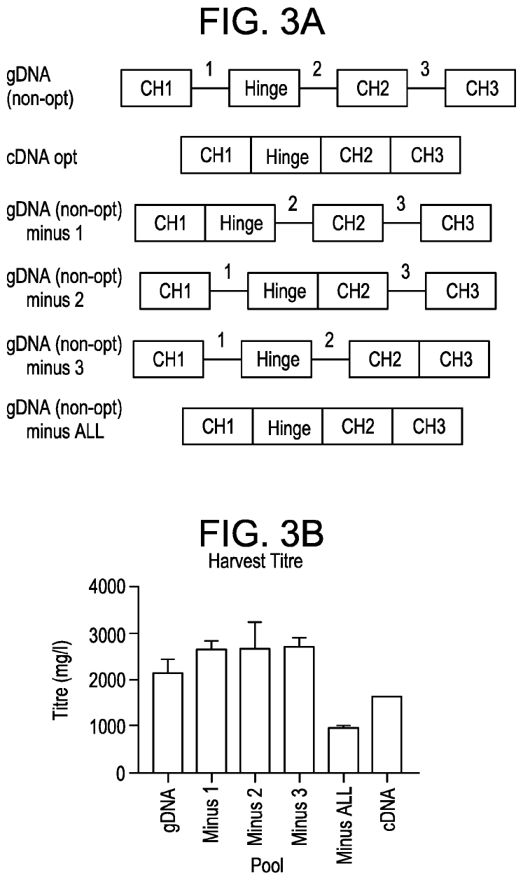

Because the two immunoglobulin fragment variants result from mis-splicing in intron 2, the individual introns were removed from the immunoglobulin heavy chain constant region and compared to cDNA versions or gDNA versions without any immunoglobulin heavy chain constant region introns. The following constructs were created and tested: (1) gDNA (non-codon optimized) containing all three introns in the heavy chain constant region; (2) cDNA (codon optimized); (3) gDNA (non-codon optimized) with intron 1 removed; (4) gDNA (non-codon optimized) with intron 2 removed; (5) gDNA (non-codon optimized) with intron 3 removed; and (6) gDNA (non-codon optimized) with all introns removed. See FIG. 3A.

The nucleic acids were prepared and transfected in CHO cells as described in Example 1 to the pool stage. The pools were screened for single cell clones by using a Single Cell Printer™ (Cytena), which deposits droplets containing single cells into a well of a 384-well plate. Single cell deposition is confirmed using a Cellavista® plate reader (Synentec). The top expressing clone was selected for further characterization. The top clones were grown and induced to produce MAb2 for each construct. MAb2 was harvested on Day 13. FIG. 3B demonstrates that the harvest titre for the cDNA and gDNA (with no introns) had the lowest harvest titer. While the gDNA minus one of either intron 1, intron 2, or intron 3 constructs resulted in increased harvest titer compared to gDNA containing all three introns, gDNA (with no introns), and cDNA constructs.

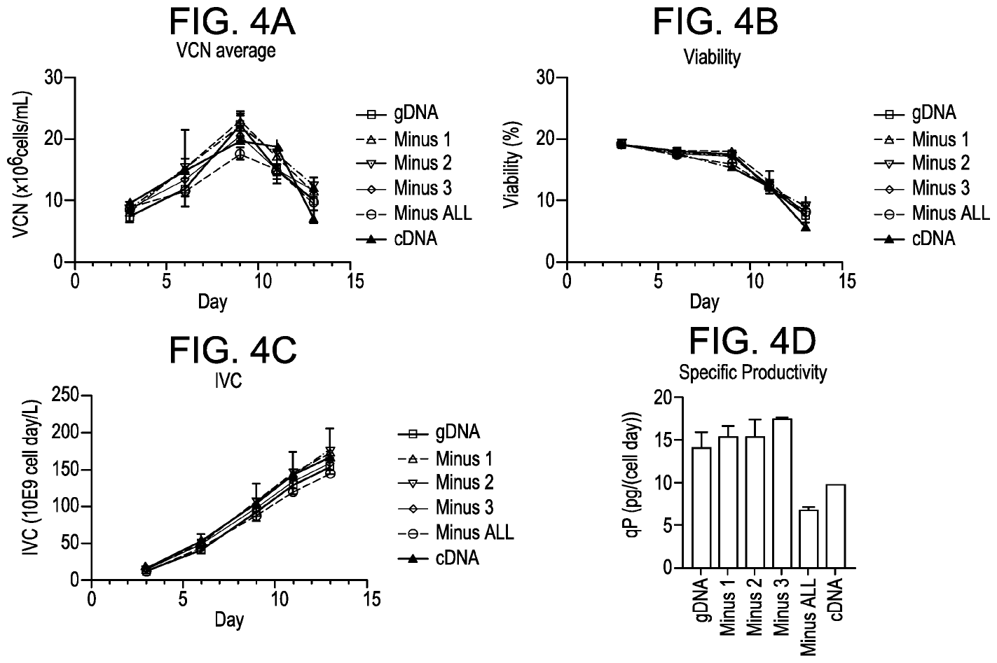

FIGS. 4A-C demonstrate that the average viable cell number (VCN), cell viability, and integral of viable cells (IVC) were approximately the same between all constructs, but FIG. 4D reveals that the increase in titer comes from increase in cell productivity (qP). Since it was known that introns increase immunoglobulin expression, it was surprising that removing one intron increased harvest titer.

### Example 3. Engineering Nucleic Acids to Increase Harvest Titer

To further determine the importance of each intron on the ability of the CHO cells to produce an immunoglobulin, the following MAb2 constructs were created: (1) gDNA (non-codon optimized) with intron 2 removed; (2) gDNA (non-codon optimized) with introns 1 and 2 removed; (3) gDNA (non-codon optimized) with introns 2 and 3 removed; and (4) gDNA (non-codon optimized) without any introns. To determine the importance of using codon-optimized nucleotide sequences, the same set of constructs were created except using codon-optimized sequences.

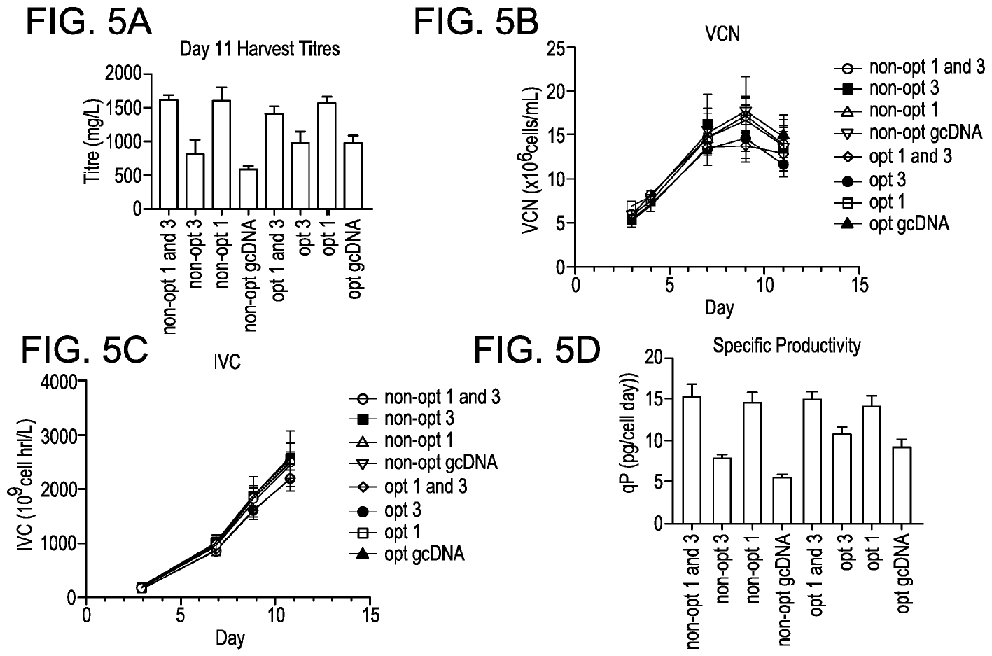

The construct was transfected into CHO cells, as in Example 2. Similarly, the CHO cells were grown and induction of immunoglobulin production was followed as in Example 2. Day 11 the immunoglobulin products were harvested and harvest titer, qP, VCN, and IVC were all determined. FIG. 5A reveals that harvest titers for immunoglobulin produced from gDNA without intron 2 and gDNA without introns 2 and 3 were the highest, regardless if the nucleotide sequence was codon-optimized or not. However, gDNA without introns 1 and 2 had similar titer levels to gDNA without any introns. FIGS. 5B and 5C demonstrate that VCN and IVC are approximately the same for each. However, cell productivity (qP) revealed that gDNA without intron 2 and gDNA without introns 2 and 3 were the most productive. See FIG. 5D. This suggests that intron 1 is important in increasing production of immunoglobulin.

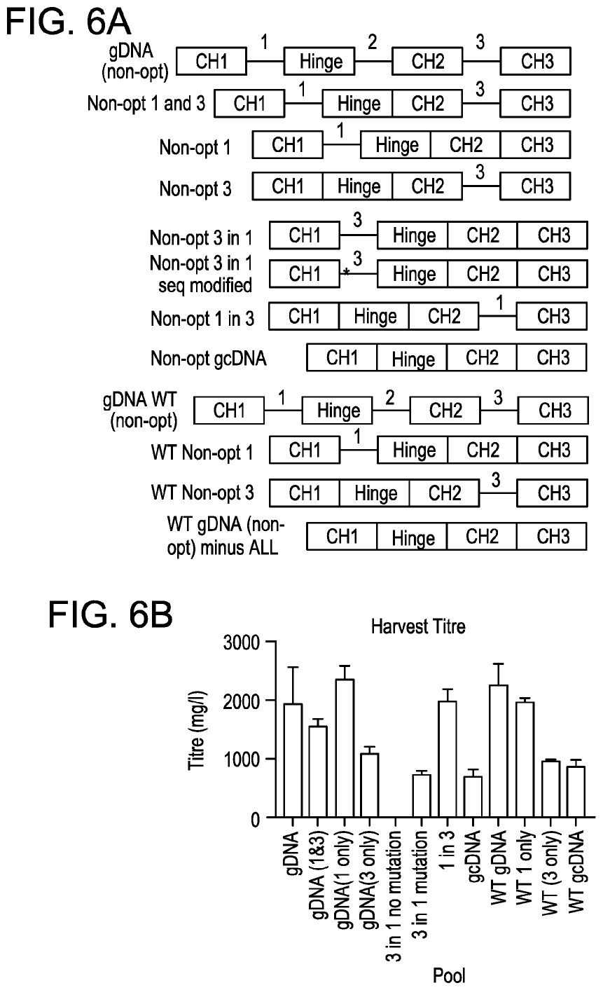

To further understand the importance of intron 1 on increasing immunoglobulin production, several new MAb2 constructs were created. In addition to the non-codon optimized constructs created above (i.e., gDNA (non-codon optimized) with all introns, gDNA (non-codon optimized) without intron 2, gDNA (non-codon optimized) without introns 2 and 3, and gDNA (non-codon optimized) without introns 1 and 2), the following constructs were created: (1) gDNA (non-codon optimized) with intron 3 moved to the location of intron 1 and deleting introns 1 and 2; (2) gDNA (non-codon optimized) with intron 3 moved to the location of intron 1, the nucleotide sequence of intron 3 was modified to increase the strength of the 5′ splice donor site by making a 1 nucleotide change to the intron sequence, and deleting introns 1 and 2; (3) gDNA (non-codon optimized) with intron 1 moved to the location of intron 3, and deleting introns 2 and 3; (4) gDNA (non-codon optimized) without any introns; (5) gDNA (non-codon optimized) with wild type IgG1 and all introns; (6) gDNA (non-codon optimized) with wild type IgG1 without introns 2 and 3; (7) gDNA (non-codon optimized) with wild type IgG1 without introns 1 and 2; and (8) gDNA (non-codon optimized) with wild type IgG1 without any introns. See FIG. 6A.

These constructs were transfected into CHO cells as described in Example 1. The CHO cells were grown and induced to produce immunoglobulin as described in Example 1. The immunoglobulin was harvested on Day 11 and the harvest titer was determined. FIG. 6B demonstrates that moving intron 1 into the location of intron 3 results in similar harvest titers as gDNA with all introns and gDNA with only intron 1. FIG. 6B also confirms that the effect of intron 1 in maintaining a similar titre as a construct containing all three introns is not influenced by whether the format is wild type IgG1 or half-life extended IgG1.

### Example 4. Engineering Nucleic Acids in Additional Immunoglobulins to Determine if there is Increased Harvest Titer

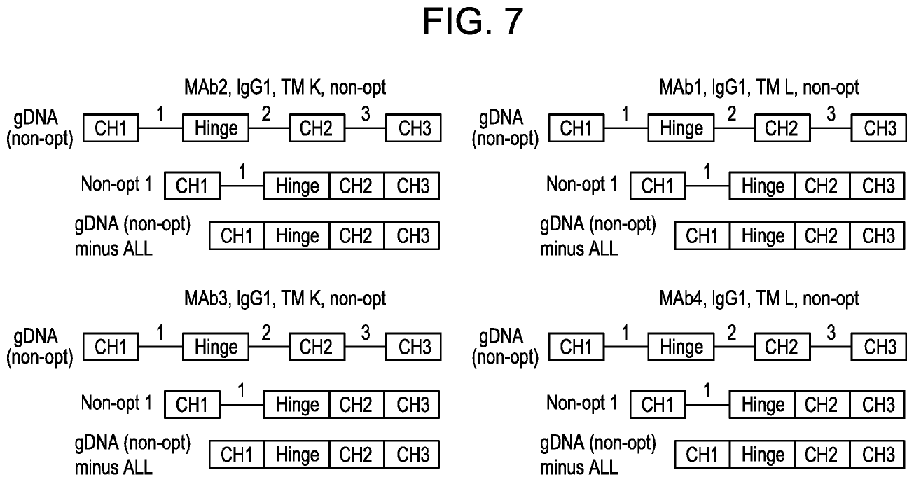

To determine if the results seen in MAb2 are specific to that immunoglobulin, additional immunoglobulin molecules are tested. FIG. 7 shows the different constructs created for MAb2, MAb1, MAb3, and MAb4. For each immunoglobulin the following constructs were created: (1) gDNA (non-codon optimized) with all introns; (2) gDNA (non-codon optimized) with only intron 1; and (3) gDNA (non-codon optimized) without any introns. MAb2 and MAb3 contain a kappa light chain, while MAb1 and MAb4 contain a lambda light chain. The lambda light chain has a different intron between the variable light chain and constant light chain, and different polyA tail. These constructs were created and as previous described in Example 1. These constructs were transfected into CHO cells as described in Example 2 to generate pools. The CHO cells were grown and induced to produce immunoglobulin as described in Example 2. The immunoglobulin was harvested on Day 11 and the harvest titer was determined.

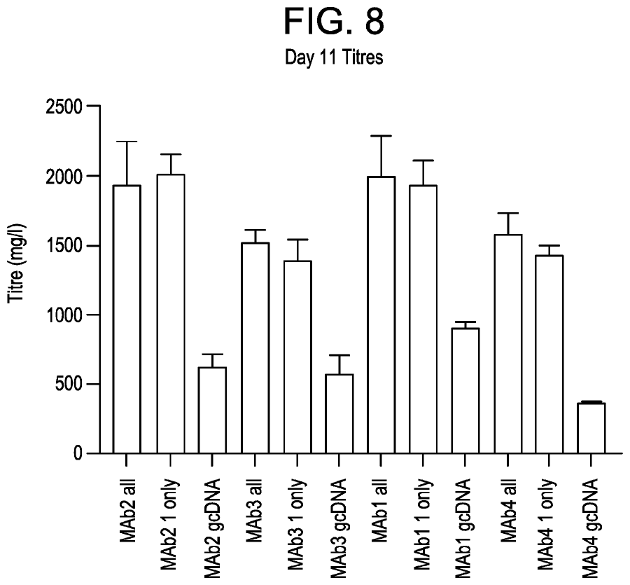

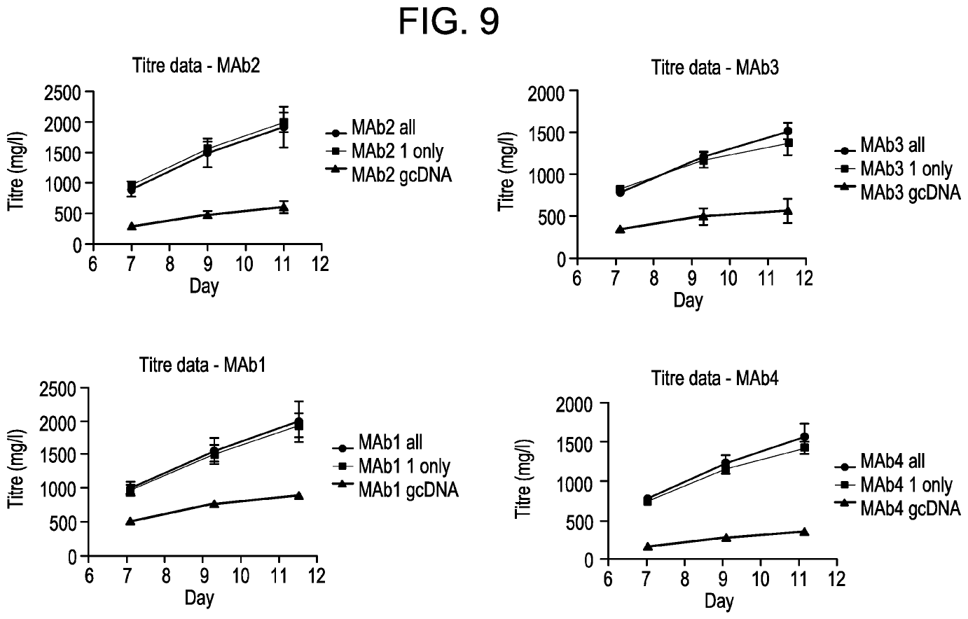

FIG. 8 demonstrates that the presence of only intron 1 in the heavy chain constant region of MAb2, MAb3, MAb1, and MAb4 results in similar immunoglobulin titers compared to each construct having all heavy chain constant region introns. Additionally, the constructs having only intron 1 had increased titers compared to gDNA without any introns in the immunoglobulin heavy chain constant region. Moreover, FIG. 9 shows that immunoglobulin titer from Day 7 to Day 11 is similar between constructs with all heavy chain constant region introns as compared to constructs with only intron 1 in the heavy chain constant region for MAb2, MAb3, MAb1, and MAb4. For all molecules tested, the gDNA without any introns had significantly lower immunoglobulin titers than the construct with all introns in the heavy chain constant region and intron 1-only construct.

These data provide further evidence that intron 1 of the heavy chain constant region is important in maintaining higher immunoglobulin titer levels. Moreover, this data shows that the increased titer levels are not limited to MAb2. Moreover, the presence of an immunoglobulin kappa or lambda light chain does not affect titer levels.

### Example 5. Engineering Nucleic Acids Replace Intron 3 with an Intron the Same Size as Intron 1

To determine why intron 1 can increase harvest titer, this Example will demonstrate that it is the size of intron 1 rather than the nucleotide sequence itself. Intron 1 in MAb2 is 391 nucleotides, while intron 3 is 97 nucleotides. The following constructs will be created: (1) MAb2 gDNA (non-codon optimized) with all introns; (2) MAb2 (non-codon optimized) without introns 2 and 3; (3) MAb2 (non-codon optimized) without introns 1 and 2, but increasing the number of nucleotides in intron 3 to be approximately the same size as intron 1; (4) MAb2 (non-codon optimized), but reducing the size of intron 1 to approximately the size of intron 3, and deleted introns 2 and 3; and (5) MAb2 gDNA (non-codon optimized) without any introns.

These constructs will be created and as previous described in Example 2. These constructs will be transfected into CHO cells as described in Example 2. The CHO cells will be grown and will be induced to produce immunoglobulin as described in Example 1. The immunoglobulin will be harvested on Day 11 and the harvest titer will be determined. The results will show that increasing the size of intron 3 will cause an increase in harvest titer and be similar to MAb2 without introns 2 and 3. However, decreasing the size of intron 1 will result in similar results as MAb2 without introns 1 and 2. This Example will reveal that the size of intron 1 is important, rather than the nucleotide sequence itself.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other aspects are within the following claims.

What is claimed:

1. An isolated nucleic acid comprising a nucleotide sequence encoding an immunoglobulin heavy chain, wherein the nucleotide sequences of intron 2 and intron 3 of the immunoglobulin heavy chain constant region are deleted.

2. An isolated nucleic acid comprising a nucleotide sequence encoding an immunoglobulin heavy chain wherein the nucleotide sequence of one intron of the immunoglobulin heavy chain constant region is deleted.

3. The nucleic acid of claim 2, wherein the nucleotide sequence of intron 1 of the immunoglobulin heavy chain constant region is deleted.

4. The nucleic acid of claim 2, wherein the nucleotide sequence of intron 2 of the immunoglobulin heavy chain constant region is deleted.

5. The nucleic acid of claim 2, wherein the nucleotide sequence of intron 3 of the immunoglobulin heavy chain constant region is deleted.

6. An isolated nucleic acid comprising a nucleotide sequence encoding an immunoglobulin heavy chain, wherein the nucleotide sequences of two introns of the immunoglobulin heavy chain constant region are deleted.

7. The nucleic acid of claim 6, wherein the nucleotide sequences of intron 1 and intron 2 of the immunoglobulin heavy chain constant region are deleted.

8. The nucleic acid of claim 6, wherein the nucleotide sequences of intron 1 and intron 3 of the immunoglobulin heavy chain constant region are deleted.

9. An isolated nucleic acid comprising a nucleotide sequence encoding an immunoglobulin heavy chain, wherein the nucleotide sequence of intron 1 of the immunoglobulin heavy chain constant region is deleted and the nucleotide sequence of intron 2 and/or intron 3 of the immunoglobulin heavy chain constant region are deleted, and wherein the nucleotide sequences of intron 2 and/or intron 3 are substituted with the nucleotide sequence of intron 1.

10. The nucleic acid of claim 9, wherein the nucleotide sequence of intron 2 of the immunoglobulin heavy chain constant region is substituted with the nucleotide sequence of intron 1.

11. The nucleic acid of claim 9, wherein the nucleotide sequence of intron 3 of the immunoglobulin heavy chain constant region is substituted with the nucleotide sequence of intron 1.

12. An isolated nucleic acid comprising a nucleotide sequence encoding an immunoglobulin heavy chain, wherein the nucleotide sequences of intron 2 and/or intron 3 of the immunoglobulin heavy chain constant region are replaced with the nucleotide sequence of intron 1.

13. The nucleic acid of claim 12, wherein the nucleotide sequence of intron 2 of the immunoglobulin heavy chain constant region is replaced with the nucleotide sequence of intron 1.

14. The nucleic acid of claim 12, wherein the nucleotide sequence of intron 3 of the immunoglobulin heavy chain constant region is replaced with the nucleotide sequence of intron 1.

15. An isolated nucleic acid comprising a nucleotide sequence encoding an immunoglobulin heavy chain, wherein the nucleotide sequence of intron 3 of the immunoglobulin heavy chain constant region is replaced with a nucleotide sequence of an intron comprising about the same number of nucleotides as the nucleotide sequence of intron 1 of the immunoglobulin heavy chain constant region.

16. An isolated nucleic acid comprising a nucleotide sequence encoding an immunoglobulin heavy chain, wherein the nucleotide sequence of intron 2 of the immunoglobulin heavy chain constant region is replaced with a nucleotide sequence of an intron comprising about the same number of nucleotides as the nucleotide sequence of intron 1 of the immunoglobulin heavy chain constant region.

17. The nucleic acid of claim 15 or 16, wherein the nucleotide sequence of intron 1 of the immunoglobulin heavy chain constant region is deleted.

18. The nucleic acid of any one of claims 1-17, wherein the nucleic acid expresses an immunoglobulin at a higher titer than a nucleic acid containing all intron sequences of the immunoglobulin heavy chain constant region when expressed with a nucleic acid encoding an immunoglobulin light chain.

19. The nucleic acid of any one of claims 1-17, wherein the nucleic acid expresses an immunoglobulin at a higher titer than a nucleic acid containing no intron sequences of the immunoglobulin heavy chain constant region when expressed with a nucleic acid encoding an immunoglobulin light chain.

20. The nucleic acid of claim 18 or 19, wherein the immunoglobulin light chain is a kappa light chain or lambda light chain.

21. The nucleic acid of any one of claims 1-20, wherein the nucleic acid is codon optimized.

22. The nucleic acid of any one of claims 18-21, wherein the expressed immunoglobulin has an IgG1, IgG2, IgG3, or IgG4 isotype.

23. The nucleic acid of claim 22, wherein the immunoglobulin is a human, humanized, chimeric, or resurfaced immunoglobulin.

24. The nucleic acid of any of claims 1-23, which is a deoxyribonucleic acid (DNA).

25. A vector comprising the nucleic acid of any one of claims 1-24.

26. An expression vector comprising the nucleic acid of any one of claims 1-25.

27. A host cell comprising the vector of claim 25.

28. A host cell comprising the expression vector of claim 26.

29. The host cell of claim 27 or 28, wherein the host cell is a eukaryotic cell.

30. The host cell of claim 29, wherein the eukaryotic cell is a Chinese Hamster Ovary (CHO) cell.

31. A method of producing an immunoglobulin, comprising culturing a host cell in a medium under conditions in which the cell expresses the immunoglobulin; wherein the host cell comprises the immunoglobulin heavy chain-encoding nucleic acid of claim 1 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain.

32. A method of producing an immunoglobulin, comprising culturing a host cell in a medium under conditions in which the cell expresses the immunoglobulin; wherein the host cell comprises the immunoglobulin heavy chain-encoding nucleic acid of claim 2 and a nucleic acid encoding an immunoglobulin light chain, wherein the host cell expresses the immunoglobulin at a higher titer than a host cell comprising a nucleic acid encoding an immunoglobulin heavy chain comprising all or none of introns 1-3 of the immunoglobulin heavy chain constant region are present, and a nucleic acid encoding an immunoglobulin light chain.

33. The method of claim 32, wherein the nucleotide sequence of intron 1 of the immunoglobulin heavy chain constant region is deleted.

34. The method of claim 32, wherein the nucleotide sequence of intron 2 of the immunoglobulin heavy chain constant region is deleted.

35. The method of claim 32, wherein the nucleotide sequence of intron 3 of the immunoglobulin heavy chain constant region is deleted.

36. A method of producing an immunoglobulin, comprising culturing a host cell in a medium under conditions in which the cell expresses the immunoglobulin; wherein the host cell comprises the immunoglobulin heavy chain-encoding nucleic acid of claim 6 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain.

37. The method of claim 36, wherein introns 1 and 2 of the immunoglobulin heavy chain constant region are deleted.

38. The method of claim 36, wherein introns 1 and 3 of the immunoglobulin heavy chain constant region are deleted.

39. A method of producing an immunoglobulin, comprising culturing a host cell in a medium under conditions in which the cell expresses the immunoglobulin; wherein the host cell comprises the immunoglobulin heavy chain-encoding nucleic acid of claim 9 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 the immunoglobulin heavy chain constant region are present, and a nucleic acid encoding an immunoglobulin light chain.

40. The method of claim 39, wherein the nucleotide sequence of intron 2 of the immunoglobulin heavy chain constant region is substituted with the nucleotide sequence of intron 1.

41. The method of claim 39, wherein the nucleotide sequence of intron 3 of the immunoglobulin heavy chain constant region is substituted with the nucleotide sequence of intron 1.

42. A method of producing an immunoglobulin, comprising culturing a host cell in a medium under conditions in which the cell expresses the immunoglobulin; wherein the host cell comprises the immunoglobulin heavy chain-encoding nucleic acid of claim 12 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 the immunoglobulin heavy chain constant region are present, and a nucleic acid encoding an immunoglobulin light chain.

43. The method of claim 42, wherein the nucleotide sequence of intron 2 of the immunoglobulin heavy chain constant region is replaced with the nucleotide sequence of intron 1.

44. The method of claim 42, wherein the nucleotide sequence of intron 3 of the immunoglobulin heavy chain constant region is replaced with nucleotide sequence of intron 1.

45. A method of producing an immunoglobulin, comprising culturing a host cell in a medium under conditions in which the cell expresses the immunoglobulin; wherein the host cell comprises the immunoglobulin heavy chain-encoding nucleic acid of claim 15 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain.

46. A method of producing an immunoglobulin, comprising culturing a host cell in a medium under conditions in which the cell expresses the immunoglobulin; wherein the host cell comprises the immunoglobulin heavy chain-encoding nucleic acid of claim 16 and a nucleic acid encoding an immunoglobulin light chain, wherein the host cell expresses the immunoglobulin at a higher titer than a host cell comprising a nucleic acid encoding an immunoglobulin heavy chain wherein all or none of introns 1-3 of the immunoglobulin heavy chain constant region are present, and a nucleic acid encoding an immunoglobulin light chain.

47. The method of claim 45 or 46, wherein the nucleotide sequence of intron 1 in the immunoglobulin heavy chain constant region is deleted.

48. The method of any one of claims 31-47, wherein the immunoglobulin heavy chain-encoding nucleic acid is a deoxyribonucleic acid (DNA).

49. The method of any one of claims 31-47, wherein the immunoglobulin light chain is a kappa light chain or lambda light chain.

50. The method of any one of claims 31-49, wherein the immunoglobulin heavy chain-encoding nucleic acid is codon optimized.

51. The method of any one of claims 31-50, wherein the expressed immunoglobulin has an IgG1, IgG2, IgG3, or IgG4 isotype.

52. The method of claim 51, wherein the expressed immunoglobulin is a human, humanized, chimeric, or resurfaced immunoglobulin.

53. The method of any one of claims 31-52, wherein the expressed immunoglobulin produced from a pool of clones has a harvest titer of at least 1,000 mg/L, of at least 1,500 mg/L, of at least 2,000 mg/L, of at least 2,500 mg/L, or of at least 3,000 mg/L.

54. The method of any one of claims 31-53, wherein the immunoglobulin produced from a top expressing clone has a harvest titer of at least 1,000 mg/L, of at least 1,500 mg/L, of at least 2,000 mg/L, of at least 3,000 mg/L, of at least 4,000 mg/L, of at least 5,000 mg/L, of at least 6,000 mg/L, of at least 7,000 mg/L, of at least 8,000 mg/L, of at least 9,000 mg/L, of at least 10,000 mg/L, of at least 11,000 mg/L, or of at least 12,000 mg/L.

55. The method of any one of claims 31-54, wherein the host cell is a eukaryotic cell.

56. The method of claim 55, wherein the eukaryotic cell is a CHO cell.
